(19) United States
(12) Patent Application Publication
(10) Pub. No.: US 2010/0048457 A1
(43) Pub. Date: Feb. 25, 2010

(54) GLYCOPROTEIN FOR TREATING CHRONIC OBSTRUCTIVE PULMONARY DISEASES

(75) Inventor: Jinkui Xie, Guangdong (CN)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: Guangzhou Konzern Pharmacetuical Co. Ltd, Guangdong (CN)

(21) Appl. No.: 12/526,055

(22) PCT Filed: Jan. 17, 2008
(86) PCT No.: PCT/CN2008/070124

§ 371 Date: Aug. 6, 2009

(30) Foreign Application Priority Data
Feb. 6, 2007 (CN) 200710026751.0

(51) Int. Cl. A61K 38/16; A61P 11/00; A61P 31/04

(52) U.S. Cl. 514/8

(57) ABSTRACT

Provided is a glycoprotein product extracted from limax, wherein the preparation method of the glycoprotein product comprises the following steps: adding ethanol to the dry limax powders and removing the supernatant, extracting the resulting residue with water and then adding ethanol to the water extract for precipitating. The obtained glycoprotein product can be used for treating chronic obstructive pulmonary diseases (COPD) and has antibacterial and antiphlogistic effects.

### TECHNICAL FIELD

The present invention relates to a new pharmaceutical composition for the treatment of chronic obstructive pulmonary diseases, particularly relates to a glycoprotein extract which is extracted from limax material.

### BACKGROUND ART

Chronic obstructive pulmonary diseases (COPD) are generally known as chronic airway obstruct diseases, including two main diseases: chronic bronchitis and emphysema with irreversible airway obstruct. Newly national investigation of COPD was carried out from 2002, mastermind by Chinese Society of Respiratory Diseases of Chinese Medical Association, to 2006 for four years. 20,245 persons aged 40 or more, selected from cities and villages in Guangdong, Beijing, and Shanghai, etc., had been investigated through questionnaire, physical examination, and functional test of lung, and found that the total suffering ratio of COPD is 8.2%, which is lower than the average level of the world, 10%. The suffering ratio of male persons is 12.4%, and the suffering ratio of female persons is 5.1%. The suffering ratio of persons in cities is 8.8%, and the suffering ratio of persons in villages is 7.8%. Now there are about 40 million sufferers of COPD in China, and there are more than 1 million sufferers of COPD died every year. There are about 5 to 10 million sufferers of COPD became disabled. In these sufferers, only 65.8% have obvious symptom, most of sufferers are ignored due to their non-obvious symptom. Up to now, there are only about 30% sufferers of COPD had been diagnosed and treated, and the 70% sufferers have been ignored. In the past 35 years, the mortality rate of COPD has raised 100%, COPD becomes the fourth “killer” to persons in cities, and the other three “killers” are stroke, cancer, and cardiopathy.

There are 15% to 20% active smokers, particularly smoke for many years, may become sufferers of COPD, and the suffering ratio of passive smokers have been raised 10% to 43%. Persons repeatedly suffering respiratory tract diseases, persons suffering indoor pollution, and persons working in pulverous environment are high risk persons who are easily induced to COPD. The suffering ratio and the mortality rate of COPD will continue raise in the future. So the prevention and cure of COPD have been drawn attention to the modem society.

The main drug for COPD is bronchia dilatation reagents, which can improve the symptom of sufferers, however, the effect of the bronchia dilatation reagent is weak. Inhalant anticholinergic drugs is stronger than short-term inhalant β2 receipt agonist in the effect of dilatation of bronchia. Long-term inhalant β2 receipt agonist is normally used and effective bronchia dilatation reagent. Aminophylline is used widely for treatment of COPD, and has antiphlogistic effect.

Because there is chronic inflammation in the trachea and lung of sufferers of COPD, glucocorticoid is expected to prevent the development of COPD. However, there are only 10% sufferers of COPD have effect to glucocorticoid treatment, and they usually use the inhalant glucocorticoid due to asthma syndrome. The other sufferers of COPD have no effect to glucocorticoid treatment. Four long-term, large-scale clinical research show that glucocorticoid has no effect in treatment of COPD. Now there is no special drug or method with good effect for COPD. Thus, the new drugs for COPD need to be research and development.

### Contents of the Invention

The object of this invention is to provide a new pharmaceutical composition for the treatment of chronic obstructive pulmonary diseases (COPD).

According to the present invention, there is provided a glycoprotein extract which can be used for treating chronic obstructive pulmonary diseases, said glycoprotein mixture is prepared by the following method:

(1) crushing limax material to dry limax powders, storing for next step;

(2) extracting said dry limax powders by ethanol, then filtrating, removing the supernatant, obtaining a residue I;

(3) eliminating ethanol from said residue I, adding water to extract, then filtrating, obtaining a supernatant I;

(4) precipitating said supernatant I by ethanol, concentrating by the centrifugal, removing the supernatant, and eliminating ethanol from the deposit, freeze drying, then the targeted glycoprotein extract is obtained.

In the step (2), preferred ethanol is 60% ethanol, and the extracting process is following by: extracting said dry limax powders by 60% ethanol overnight, then filtrating, removing the supernatant, obtaining a residue I, repeating the above extracting 1-3 times by 60% ethanol in need.

In said step (4), preferred ethanol is 60% ethanol.

In the step (3), add water to extract, then filtrate, obtain a supernatant I and a residue II, and then extract the residue II by repeating the step (3), and then combine the supernatant as supernatant I.

The limax material in step (1) is selected frown fresh limax or frozen limax in whole body.

The second object of this invention is to provide a pharmaceutical composition, which comprises of effective amount of the glycoprotein extract according to the present invention, and pharmaceutically acceptable excipients, thinners, and carriers. The pharmaceutical composition is in the form of: capsule, granulate, tablet, pill, guttate pill, syrup, injection, frozen powder for injection, and spray.

The third object of this invention is to provide a use of the glycoprotein extract according to the present invention in preparation of pharmaceutical compositions for the treatment of chronic obstructive pulmonary diseases.

The fourth object of this invention is to provide a use of the glycoprotein extract according to claim 1 in preparation of antibacterial and antiphlogistic pharmaceutical compositions.

The antibacterial pharmaceutical compositions are used to preparing antibacterial agents against acute pharyngitis and/or chronic pharyngitis.

Limax (Agriolimax agrestis Linnaeus) is a mollusk animal in Gastropoda. Fresh or dry body of limax can be used as medicine. Chinese archaian medical literatures, such as Shen Nong Ben Cao Jing, and Ben Cao Gang Mu, and Chinese modem medical literatures, such as Great Thesaurus of Chinese Traditional Medicine, are all describe limax and its effect. The glycoprotein extract according to the present invention is extracted from limax material, it is proved to could be used as medicine to treat pulmonary edema and mucus related conditions.

In the preparation method of the glycoprotein extract according to the present invention, the solvents used in each steps are non-toxic ethanol and water, which could be eliminated easily, thus, the glycoprotein extract without any toxic substance is obtained. The glycoprotein extract is proved by some experimentation in vivo and in vitro that it has special good effect for COPD.

The above mentioned glycoprotein extract has the following advantages:

1. In the asthmatic guinea pig model, the number of some cells, such as white cells in the peripheral blood, neutrophilic granulocyte, particularly eosinophils, in the bronchoalveolar lavage fluid (BALF) and the pneumonic tissue, raises obviously. After feeding with the glycoprotein extract according to the present invention in certain effective amount, we analyze bronchoalveolar lavage fluid and the slice of pneumonic tissues and find that the number of all kinds of inflammation related cells, particularly eosinophils, reduces obviously, P<0.01, which shows a obvious relationship between amount and effect.

2. The glycoprotein extract according to the present invention can promote the expel of Phenolsulfonphthalein from the mucosa of trachea and bronchia, and it has the same effect of amethocholine chloride in the same concentration.

3. The glycoprotein extract according to the present invention can promote the ciliary movement in trachea mucous, and the high dosage and middle dosage of the glycoprotein extract has the same effect of acetylcholine chloride in the same concentration.

4. The glycoprotein extract according to the present invention is proved to have antibacterial and antiphlogistic effect, and it has the same effect of ofloxacin in the same concentration.

5. Our experiment shows that the lycoprotein extract according to the present invention can reduce the mortality rate of the asthmatic guinea pig model and prolong the delitescence.

The lycoprotein extract according to the present invention may be used to restrain the infiltration of the airway wall with inflammatory cells in asthmatic guinea pig model, and used for the treatment of asthma, cough, and mucus related conditions, thus used for the treatment of chronic obstructive pulmonary diseases (COPD). The principles and operation of the lycoprotein extract for treating chronic obstructive pulmonary diseases according to the present invention may be better understood with reference to the drawings and the accompanying embodiments. Accordingly, it is intended to embrace all such alternatives, modifications and variations that fall within the spirit and broad scope of the appended claims.

### BRIEF DESCRIPTION OF THE DRAWINGS

The invention is herein described, by way of example only, with reference to the accompanying drawings, wherein:

FIG. 1 is a microscopical view showing the inflammatory lesions in pneumonic tissue of bronchia of control group of mouse model in Experiment 2.5;

FIG. 2 is a microscopical view showing the inflammatory lesions in pneumonic tissue of bronchia of low dosage group feeding the lycoprotein extract of mouse model in Experiment 2.5;

FIG. 3 is a microscopical view showing the inflammatory lesions in pneumonic tissue of bronchia of middle dosage group feeding the lycoprotein extract of mouse model in Experiment 2.5;

FIG. 4 is a microscopical view showing the inflammatory lesions in pneumonic tissue of bronchia of high dosage group feeding the lycoprotein extract of mouse model in Experiment 2.5;

FIG. 5 is a microscopical view showing the inflammatory lesions in pneumonic tissue of bronchia of control group feeding dexamethason of mouse model in Experiment 2.5;

FIG. 6 is a graph showing the development of the delitescence of each experimental group in Experiment 2.6, in which, group A is asthmatic model group, group B is aminophylline treatment group, group C is low dosage treatment group, group D is middle dosage treatment group, and group E is high dosage treatment group, *P<0.01, compare with the non-treatment group;

FIG. 7 is a morphological view showing the infiltration of pneumonic tissue with eosinophils in normal control group in Experiment 2.6;

FIG. 8 is a morphological view showing the infiltration of pneumonic tissue with eosinophils in normal asthmatic model group in Experiment 2.6;

FIG. 9 is a morphological view showing the infiltration of pneumonic tissue with eosinophils in aminophylline treatment group in Experiment 2.6;

FIG. 10 is a morphological view showing the infiltration of pneumonic tissue with eosinophils in low dosage treatment group in Experiment 2.6;

FIG. 11 is a morphological view showing the infiltration of pneumonic tissue with eosinophils in middle dosage treatment group in Experiment 2.6; and

FIG. 12 is a morphological view showing the infiltration of pneumonic tissue with eosinophils in high dosage treatment group in Experiment 2.6.

### DETAILED DESCRIPTION OF THE INVENTION

### Embodiment 1: Preparation of the Glycoprotein Extract from Limax Material

Source of limax material: Vaginnlns alte Ferussac, Raise, Guangxi, China

Pretreatment: freezing the fresh limax material under −20° C., and preserving in icebox. Thaw the frozen material in the double distilled water, and remove the mud and sand from the limax material.

Preparation Method:

The cleaned limax material is divided into four groups, each is 1 kg, after thawing the frozen material in the double distilled water for about two hours. Then, the limax material is crushed to dry limax powders by high-speed tissue crusher, such as DS-200 crusher of Jiangsu Jiangyin Instrument Factory, each time 200 g, 5 times, then concentrated by the centrifugal in 4000 r/min for 10 minutes in order to separated the supernatant and residue. Repeat the above steps, finally add 1 L, 2 L, 3L, and 4 L double distilled water into the resulting residue. The solution of residue is kept in 4° C. overnight, then filtrated by four-layers gauze, and then suction filtrated by filter papers, determine the quantity of glycoprotein by phenol-vitriol method, and determine the quantity of protein by BCA method.

The extract solution will be carried out by the following freeze dry procedure: the filtrated fluid is put into a freeze dryer in −40° C. for 12 hours, raise the temperature gradually to 4° C., and keep in 4° C. for 12 hours, and then raise the temperature gradually from 4° C. to 30° C., the whole process stands 10 hours, the glycoprotein extract will be obtained finally.

### Embodiment 2: Preparation of Forms of Pharmaceutical Composition Comprising of the Glycoprotein Extract

Mix the glycoprotein extract prepared in example I and certain supplementary materials, the mixtures is made into the form of: capsule, granulate, tablet, pill, guttate pill, syrup, injection, frozen powder for injection, and spray, according to the method and requirement of Chinese Pharmacopoeia 2000 first section.

### Experiments

### 1. Experimental Materials

### 1.1 Medicine and Reagent

1) Glycoprotein extract from limax (medicine): provided by pharmaceutical and pharmacology institute of University of South China. Expected human clinic amount is 700 mg, batch no. is 2005012. The sample of the glycoprotein extract is light yellow powders, slight fishy smell, and sight bitter and salty. The dosage is 10 ml/kg. The medicine is soluted in 0.9% physiological saline, restored in icebox, and shake up before using.

The dosage for animals is determined by the three times of the dosage of human considering the weight. High dosage is three times more than the middle dosage, and the middle dosage is three times more than the low dosage. The high dosage and administer concentration for rat is 210 mg/kg and 21 mg/kg. The middle dosage and administer concentration for rat is 70 mg/kg and 7 mg/kg. The low dosage and administer concentration for rat is 23 mg/kg and 2.3 mg/kg. The administer volume for rat is 10 ml/kg. The high dosage and administer concentration for mouse is 300 mg/kg and 30 mg/kg. The middle dosage and administer concentration for mouse is 100 mg/kg and 10 mg/kg. The low dosage and administer concentration for mouse is 33 mg/kg and 3.3 mg/kg. The administer volume for mouse is 10 ml/kg. The administer volume for rat is 10 ml/kg. The high dosage and administer concentration for guinea pig is 210 mg/kg and 21 mg/kg. The middle dosage and administer concentration for guinea pig is 70 mg/kg and 7 mg/kg. The low dosage and administer concentration for guinea pig is 23 mg/kg and 2.3 mg/kg. The administer volume for guinea pig is 10 ml/kg. The medicine is soluted in 0.9% physiological saline, restored in icebox, and shake up before using.

2) Ofloxacin: provided by Shanghai Pu Kang Pharmaceutical Co., Ltd., batch no. is 20060219.

3) Culture medium: beef extracts, provided by Beijing Ao Bo Xing Bio-tech Co., Ltd., batch no. is 20040218.

4) Culture medium: Tryptone, provided by Beijing Ao Bo Xing Bio-tech Co., Ltd., batch no. is 20060402.

5) Bacteriun: Staphyloccocus aureus Rosenbach, provided by Microbiology Department of University of South China; α-hemolytic streptococcus, provided by National Institute for the Control of Pharmaceutical and Biological Products.

6)Ammonium chloride: provided by Beijing Pharmaceutical Factory, batch no. is 021018.

7) Phenolsulfonphthalein: provided by Beijing Chemical Factory, batch no. is 980820.

8) Sodium bicarbonate: provided by Beijing Chemical Factory, batch no. is 031012.

9) Ovalbumin: provided by Sigma, batch no. is 90562745.

10) Aminophylline, sodium hydroxide, pentobarbital, physiological saline, Locke's solution, and sulfur dioxide: provided by Li Ke Pharmaceutical Co., Ltd.

11) IL-2, IL-4 assaying box for ELISA: provided by Shenzhen Jing Mei Co., Ltd.

12) Acetylcholine chloride: provided by Shanghai Sinopharm Chemical Reagent Co., Ltd., batch no. is 040910.

### 1.2 Experimental Animal

1) SD white rat: half is male, and half is female. Each weight is 150-200 g. Provided by Experimental Animal Center of Animal Technology College of Hunan Agricultural University, certification no. is scxk 2003-0003.

2) Kunming Mouse: half is male, and half is female. Each weight is 18-22 g. Provided by Experimental Animal Center of Animal Technology College of Hunan Agricultural University, certification no. is scxk 2003-0003.

3) Guinea pig: male or female. Each weight is 250-300 g. Provided by Experimental Animal Center of Animal Technology College of Hunan Agricultural University, certification no. is scxk 2003-0003.

4) New Zealand Rabbit: clean grade, male or female. Each weight is 1.8-2.2 kg. Provided by Experimental Animal Center of Animal Technology College of Hunan Agricultural University, certification no. is scxk 2003-0003.

5) Syrian golden hamster: male. Each weight is 100-150 g. Provided by Experimental Animal Center of Animal Technology College of Hunan Agricultural University, certification no. is scxk 2003-0003.

All above animals are feed in the lab meet the requirement of National Standard for Experimental Animal, in 20-25° C., 55%-70% humidity.

Mouse feed is provided by Experimental Animal Center of Animal Technology College of Hunan Agricultural University.

### 1.3 Main Instruments

1) BECKMAN Automatic Chemistry Analyzer: provided by BECKMAN (US).

2) OLYMPUS-CH photomicrography system: provided by OLYMPUS (JP).

3) FA1004 electronic balance: provided by Shanghai Balance Instrument Factory.

4) Type 1512 microtome: provided by Leica (DE).

5) Type 402A1 ultrasonic atomizer, lavage machine, and T pipe: provided by Jiangsu Yuyue Medical Equipment Inc.

### 2. Method and Result

2.1 Glycoprotein Extract from Limax for Treatment of Experimental Emphysema and Pulmonary Hypertension

Preparation of model animal with emphysema and pulmonary hypertension: choose 75 from 90 male golden hamster randomly, 100-150 g each, to make model mouse. The model-building is performed on elected 75 animals under intraperitoneal anesthesia. Then open the mouse and exposure its throat, insert a pipe and inject the medicine fluid (60 U/100 g weight) into the body through the pipe, then shake the animal to let the medicine equality. Animals are wake in several hours, and feed normally. Other 15 golden hamster are acted as normal control group (Group A), the same volume of saline is injected in each trachea of each animal.

After 21 days, 75 model animals are divided into five groups: model control group (Group B), positive medicine (aminophylline) control group (Group C), glycoprotein extract from limax in low dosage (23 mg/kg/d) treatment group (Group D), middle dosage (70 mg/kg/d) treatment group (Group B), high dosage (210 mg/kg/d) treatment group (Group F). After perfusion of the medicine by gavage, animals are killed to measure the following:

Right Systolic Pressure (RSP): animals are cut in the neck to expose the right jugular vein, then insert the pipe into the right jugular vein to right ventricle, the pipe also connects the physiological monitor (RM-6300, Japan) for blood pressure measuring. The right carotic artery is also exposed and cannulated for blood pressure measuring.

Right Ventricle Hypertrophy Index (RVHI): kill the animal by bleeding, open the chest and bring the heart out, then cut the right ventricle down. Wash the heart by saline, absorb the water by filter paper, and then weight the right ventricle, and weight the total weight of the left ventricle and atrioventricular septalium. RVHI=weight of right ventricle/(total weight of left ventricle and atrioventricular septalium)

Pneumonic bronchoalveolar lavage fluid analyse: isobarical lavage machine is infused with Locke's solution, 37° C., the level of bottom is 60-100 cm higher than the T pipe. Kill the animal and open its chest, separate and cut down the trachea, and then bring out the trachea and the lung together, place in the Locke's solution, 37° C. Extrude the air out of the lung, and ligate the trachea with sleeve and connect with the T pipe. Open the T pipe to let Locke's solution flow into the lung, adjust the velocity of flow to about 25 ml/min. Concentrate the lavage 400 g by the centrifugal for 10 minutes, remove the supernatant, then add physiological saline into the deposit to make the cell suspend. Count the number of cells of bronchoalveolar lavage fluid (HALF) by inverted microscope, four times for one sample. Count the number of cells (/ml/g) of bronchoalveolar lavage fluid (BALF). Dye the smear of the suspend with HE, then count the ratio of macrophage in cell types.

Statistic treatment: data is expressed by x±s, and analyzed by one-way ANOVA, and compare with each group by Newman-Keuls method, P<0.05 meants distinct difference.

### Result of Experiment:

Pathology observations and morphometric analysis:

Normal group: the structure of bronchia, alveolus, and lung vascular is normal.

Model group: there is obviously expansion in most of bronchiole, respiratory bronchiole, alveolar pipe, alveolar sac, and alveolus.

Aminophylline group: comparing with the model group, there is non-obviously improvement in pathological changes of emphysema.

Low dosage group: comparing with the model group, there is non-obviously improvement in pathological changes of emphysema.

Middle dosage group: comparing with the model group, there is certain improvement in pathological changes of emphysema, particularly, the pathological changes of arterioles are improved obviously.

High dosage group: comparing with the model group, there is certain improvement in pathological changes of emphysema, particularly, the pathological changes of arterioles are improved obviously.

Detailed data can be seen in table 1.

TABLE 1

| | MPAP (kPa) | RVHI (%) | BALF (/mm³) | MS (%) |
|---|---|---|---|---|
| Normal group | 2.79 ± 0.41 | 18.66 ± 7.34 | 77.5 ± 14.5 | 90.7 ± 2.1 |
| Model group | 5.54 ± 0.73 | 31.17 ± 6.51 | 388.2 ± 94.6 | 89.4 ± 7.3 |
| Aminophylline group | 5.07 ± 0.92 | 28.62 ± 8.81 | 364.8 ± 95.7 | 96.7 ± 9.6 |
| Low dosage group | 5.29 ± 0.85 | 30.32 ± 5.79 | 210.1 ± 87.2 | 95.7 ± 3.5 |
| Middle dosage group | 4.02 ± 0.96 | 25.55 ± 8.94 | 110.4 ± 17.9 | 95.2 ± 6.2 |
| High dosage group | 3.97 ± 0.47 | 22.27 ± 5.46 | 95.3 ± 18.8 | 91.8 ± 6.8 |

As shown in table 1, after treating 30 days, there are obviously reduction in Right Systolic Pressure (RSP), Right Ventricle Hypertrophy Index (RVHI), the number of macrophage in bronchoalveolar lavage fluid (BALF), and there is obviously improvement in pathological changes of emphysema, particularly, the pathological changes of arterioles are improved obviously (P<0.05).

2.2 Glycoprotein Extract from Limax for Treatment of Cough of Chronic Bronchitis

Preparation of model mouse with chronic bronchitis: choose 90 white mice, 20-25 g each, male or female Divide the mice randomly into six groups. In normal control group, no S02 stimulation is given, mice are feeding normally. In other groups, the following SO₂ stimulation is given: place a utensil with 0.5 g anhydrous sodium sulfite in an 8 L bell cover, and place mice of each group into the bell cover, and add 50% vitriol 5 ml into the utensil, and take out the mice after 2 minutes; Once every day, 21 days.

After 21 days, animals in model control group are given 15 ml/kg physiological saline by gavage; animals in positive control group are given 1.2 mg/kg dexamethason by gavage, once every day, 10 days, also given 15 mg/kg Dextromethorphan by gavage from the eighth day, once every day, 3 days; animals in high dosage group are given 150 mg/kg glycoprotein extract from limax by gavage, once every day, 10 days; animals in middle dosage group are given 100 mg/kg glycoprotein extract from limax by gavage, once every day, 10 days; animals in low dosage group are given 60 mg/kg glycoprotein extract from limax by gavage, once every day, 10 days. During the dosing, observe the whole condition of animals in each group, and give the above SO₂ stimulation to them in one hour after the last dosing. Record the delitescence of cough of each animal, and record the times of cough of each animal in 3 minutes. T-test among groups is made, and the comparison data among medicine groups and control groups is shown in table 2.

TABLE 2

| | Number of | | | | |
|---|---|---|---|---|---|
| Group | Animals | Dosage | | Delitescence | Times of Cough |
| Model control group | 15 | 15 | ml/kg | 12.10 ± 4.41 | 111.90 ± 10.70 |
| Dextromethorphan group | 15 | 15 | mg/kg | 17.70 ± 4.521** | 77.60 ± 9.88** |
| High dosage group | 15 | 300 | mg/kg | 16.60 ± 5.21* | 70.00 ± 14.43** |
| Middle dosage group | 15 | 100 | mg/kg | 15.90 ± 4.01* | 82.70 ± 11.67** |
| Low dosage group | 15 | 33 | mg/kg | 14.70 ± 5.17 | 105.60 ± 11.64 |

*p< 0.05,
**p < 0.01, comparing with the physiological saline control group

As shown in table 2, the glycoprotein extract from limax can obviously prolong the delitescence of cough, and reduce the times of cough. Distinct difference is shown in the high dosage group and middle dosage group, comparing with the physiological saline control group. The low dosage group tends to prolong the delitescence of cough, but has no distinct difference.

2.3 Glycoprotein Extract from Limax for Treatment of Mucus Related Conditions of Chronic Bronchitis

Preparation of model mouse with chronic bronchitis: choose 90 white mice, 20-25 g each, male or female. Divide the mice randomly into six groups. In normal control group, no SO₂ stimulation is given, mice are feeding normally. In other groups, SO₂ stimulation according to section 2.2 is given.

After 21 days, animals in model control group are given 15 ml/kg physiological saline by gavage; animals in positive control group are given 1.2 mg/kg dexamethason by gavage, once every day, 10 days, also given 15 mg/ 1.2% NH₄Cl solution by gavage from the eighth day, once every day, 3 days; animals in high dosage group are given 500 mg/kg glycoprotein extract from limax by gavage, once every day, 10 days; animals in middle dosage group are given 100 mg/kg glycoprotein extract from limax by gavage, once every day, 10 days; animals in low dosage group are given 20 mg/kg glycoprotein extract from limax by gavage, once every day, 10 days. During the dosing, observe the whole condition of animals in each group, and give 1% physiological saline and phenolsulfonphthalein to them in half of an hour after the last dosing. After half of an hour, kill the mice and put them on the table, cut and bring the trachea out of the neck, then put the trachea into 5% NH₄Cl solution, fill and flush the trachea for three times, let the solution stand overnight, and then measure the OD of the solution. The result is shown in table 3.

TABLE 3

| | Number of | | |
|---|---|---|---|
| Group | Animals | Dosage | OD value |
| Model control group | 15 | 15 ml/kg | 0.0338 ± 0.00958 |
| Dexamethason + NH₄Cl group | 15 | 1.2 mg/kg + 0.5 ml/ | 0.0736 ± 0.01106** |
| High dosage group | 15 | 500 mg/kg | 0.0640 ± 0.01376** |
| Middle dosage group | 15 | 100 mg/kg | 0.0494 ± 0.01062** |
| Low dosage group | 15 | 20 mg/kg | 0.0384 ± 0.00690 |

**p < 0.01, comparing with the physiological saline control group

As shown in table 3, the glycoprotein extract from limax can obviously increase the drainage amount of phenolsulfonphthalein from the trachea of animals. Distinct difference is shown in the high dosage group and middle dosage group, comparing with the physiological saline control group.

2.4 Anti-Asthmatic Effect of Glycoprotein Extract from Limax for Treatment of Asthma of Chronic Bronchitis

Preparation of model rat with chronic bronchitis: choose 60 SD rats, 250-300 g each, male or female. Inducing asthma by 10 ml 4% histamine phosphate and 2% acetylcholine chloride through the ultrasonic atomizer for 1 minute, the delitescence of inducing asthma is measured from starting spay to twitching, and remove the no reaction animals (over 10 minutes). Divide the pre-selected rats randomly into five groups, and build the model as following: The model-building is performed on the rats under intraperitoneal anesthesia by 0.3 ml/100 g 10% chloral hydrate. Then open the rat and exposure its trachea, insert a pipe and inject 200 ug/200 ul PLS into the trachea through the pipe, then feed for three weeks.

After 21 days, animals in model control group are given 15 ml/kg physiological saline by gavage; animals in positive control group are given 1.2 mg/kg dexamethason by gavage, once every day, 10 days, also given 0.1 g/kg aminophylline by gavage from the eighth day, once every day, 3 days; animals in high dosage group are given 210 mg/kg glycoprotein extract from limax by gavage, once every day, 10 days; animals in middle dosage group are given 70 mg/kg glycoprotein extract from limax by gavage, once every day, 10 days; animals in low dosage group are given 23 mg/kg glycoprotein extract from limax by gavage, once every day, 10 days. During the dosing, observe the whole condition of animals in each group, and measure the delitescence of inducing asthma according to above method in one hour after the last dosing. Record the result and make t-test for them. The result is shown in table 4.

TABLE 4

| | Number of | | |
|---|---|---|---|
| Group | Animals | Dosage | OD value |
| Model control group | 15 | 15 ml/kg | 292.60 ± 36.89 |
| Dexamethason + Aminophylline | 15 | 1.2 mg/kg + 0.5 ml/ | 377.00 ± 30.02** |
| group | | | |
| High dosage group | 15 | 210 mg/kg | 354.90 ± 31.00* |
| Middle dosage group | 15 | 70 mg/kg | 338.30 ± 47.95* |
| Low dosage group | 15 | 23 mg/kg | 267.80 ± 46.11 |

*p < 0.05,
**p < 0.01, comparing with the physiological saline control group

As shown in table 4, the glycoprotein extract from limax can obviously prolong the delitescence of inducing asthma by histamine phosphate and acetylcholine chloride. Distinct difference is shown in the high dosage group and middle dosage group, comparing with the physiological saline control group.

2.5 Glycoprotein Extract from Limax for Treatment of Pneumonia

Preparation of model rat with chronic bronchitis: choose 60 SD rats, 250-300 g each, male or female. Divide the rats randomly into six groups, one is normal control group, the other five groups are model groups which are modeling according to section 2.4.

After 21 days, animals in model control group are given 15 ml/kg physiological saline by gavage; animals in positive control group are given 1.2 mg/kg dexamethason by gavage, once every day, 10 days; animals in high dosage group are given 210 mg/kg glycoprotein extract from limax by gavage, once every day, 10 days; animals in middle dosage group are given 70 mg/kg glycoprotein extract from limax by gavage, once every day, 10 days; animals in low dosage group are given 23 mg/kg glycoprotein extract from limax by gavage, once every day, 10 days. During the dosing, observe the whole condition of animals in each group, and carry out the following procedures in ten days after the last dosing: The rats are narcosis under intraperitoneal anesthesia by 0.4 ml/100 g 10% chloral hydrate, and put on the operating table, open the neck and chest, bring out the trachea and pneumonic tissue, and then observe the condition of the sample. The sample is fixed by 10% formalin solution, and embedded by paraffin, and cut into slices, and dyed by HE. The inflammatory pathological changes are observed by microscope, including the integrality of epidermis, thickness of glands, infiltration of inflammatory cells in tracheal mucous, follicular lymph, and thickness of tracheal wall.

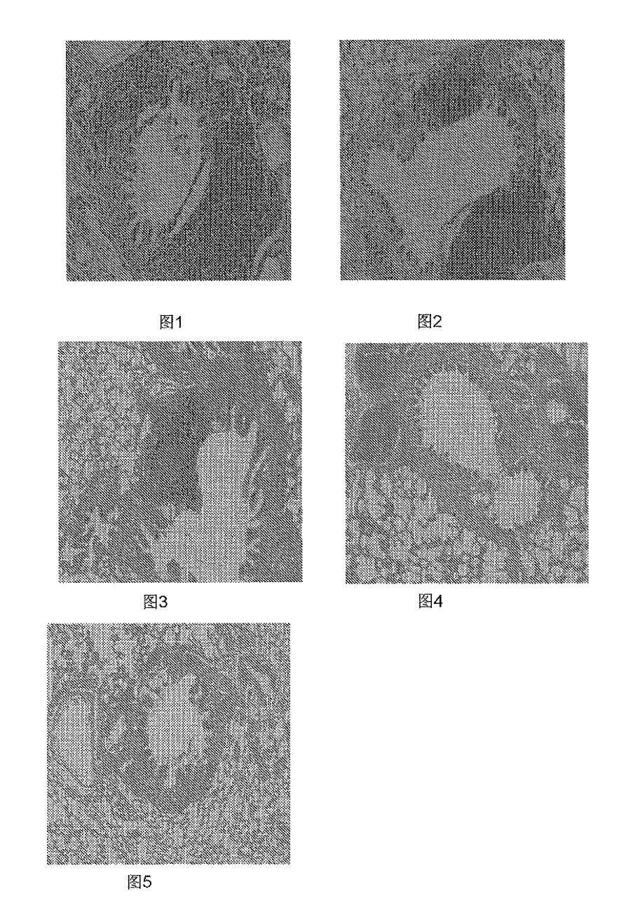

The result is that: during the 21-days adding LPS into airway, rats are tiredness, less moving, less eating food, and less drinking water. Coarse rates can be heard. After 10-days treatment, coarse rates in medicine groups are gradually reduced, and coarse rales in normal control group are not changed. Observation by eyes: pneumonic tissue expands, grey-white color, no bleeding or liquid on the surface. Observation by inverted fluorescence microscope; in model group, secretion in airway increases obviously, the epidermis is non-integrality, glands is thicker, infiltration of inflammatory cells in tracheal mucous is found, follicular lymph is formed (see FIG. 2); in treatment group, secretion in airway decreases, the epidermis is integrality, glands and tracheal wall become thinner, infiltration of inflammatory cells in tracheal mucous is smaller, bronchial peripheral follicular lymph is smaller (see FIG. 3); in the positive group (see FIG. 5) and high dosage group (see FIG. 4), they are obviously different from the model control group (see FIG. 1).

2.6 Glycoprotein Extract from Limax for Treatment of Bronchitis Asthma

Preparation of model guinea pig with bronchitis asthma: choose 90 guinea pigs, divide randomly into six groups, 15 each group. Group A: control group (healthy guinea pig); Group B: model control group (non-treatment); Group C: positive medicine control group (aminophylline); Group D: glycoprotein extract from limax capsule group (low dosage group, 23 mg/kg/d); Group E: glycoprotein extract from limax capsule group (middle dosage group, 70 mg/kg/d); Group F: glycoprotein extract from limax capsule group (high dosage group, 210 mg/kg/d).

Under sterilized conditions, each animal is injected 1 ml 100 g/L sodium hydroxide by intraperitoneal dosing, and injected 0.5 ml 1% ovalbumin (Sigma) by intramuscular injection, and control group use physiological saline instead of sodium hydroxide or ovalbumin. After two weeks, spay animals in the closed room, in isobarically 400 mmHg, with 0.5% ovalbumin in physiological saline. There are many symptom appeared related to bronchitis asthma in the treated animals. The control group is treated by the same procedure. In the next day, one hour before inducing, dose to the following groups by gavage: Group A: control group (healthy guinea pig); Group B: model control group (non-treatment); Group C: positive medicine control group (aminophylline); Group D: glycoprotein extract from limax capsule group (low dosage group, 23 mg/kg/d); Group E: glycoprotein extract from limax capsule group (middle dosage group, 70 mg/kg/d); Group F: glycoprotein extract from limax capsule group (high dosage group, 210 mg/kg/d), 7 days. The delitescence of inducing asthma is measured from spaying the ovalbumin to twitching of ventral muscles. The classification of asthma is recorded as following: divided to five classes, Class Zero: no obvious reaction; Class One: slight nose itch, tremble, and hair erection; Class Two: seldom cough, nose itch, tremble, and hair erection; Class Three: many or continue cough, breath difficulty, convulsion; Class Four: convulsion, twitch, incontinence, shock, and death. After 24 hours, animals are narcosis under intraperitoneal anesthesia by 1.5 ml 1% pentobarbital. After venous blooding, the white cells in peripheral blood are counted.

Bronchoalveolar lavage is carried out according to the pharmacological experimental method, then observe the infiltration of the pneumonic tissue with inflammatory cells, particularly eosinophils. The sample is fixed and cut into slices, and dyed by HE.

### Result of Experiment

The asthmatic guinea pig is induced for seven days, and the last inducing time is regarded as the delitescence of inducing asthma, whose unit is “s” (second). In control group, there is no obvious reaction; in asthma model group, the delitescence of inducing asthma is obviously shorten; and in medicine group, the delitescence of inducing asthma is obviously prolonged. See FIG. 6.

TABLE 5
the delitescence and mortality of each group when the experiment is finish

| | | Model | | Glycoprotein extract from | | |
|---|---|---|---|---|---|---|
| | Control | control | Aminophylline | limax capsule group | | |
| | group | group | control group | 23 mg/kg/d | 70 mg/kg/d | 210 mg/kg/d |
| Weight (g) | 470 ± 12 | 481 ± 11 | 478 ± 10 | 486 ± 14 | 485 ± 12 | 488 ± 16 |
| Delitescence | — | 87 ± 9.7 | 135 ± 15* | 127 ± 6.1* | 126 ± 8.6* | 125 ± 9.1* |
| (s) | | | | | | |
| Number of | 0 | 3 | 2 | 3 | 2 | 2 |
| death | | | | | | |

*P < 0.01, comparing with the non-treatment group

The result of number change of white cells in peripheral blood and pneumonic tissue is shown in table 6.

TABLE 6
Counting of inflammatory white cells in peripheral blood and pneumonic tissue of
each groups of guinea pigs

| | | | | Glycoprotein extract from | | |
|---|---|---|---|---|---|---|
| | | Model | | limax capsule group | | |
| | Control | control | Aminophylline | 23 mg/kg/d | 70 mg/kg/d | 210 mg/kg/d |
| | group | group | control group | (n = 13) | (n = 13) | (n = 13) |
| Peripheral | 3.6 ± 0.3 | 7.1 ± 0.5 | 3.8 ± 0.3* | 4.1 ± 0.7* | 4.0 ± 0.4* | 4.0 ± 0.4* |
| blood (×10⁹) | | | | | | |
| Bronchoalveolar | 1.6 ± 0.5 | 6.6 ± 1.3 | 1.6 ± 0.5* | 1.8 ± 0.3* | 1.5 ± 0.1* | 1.6 ± 0.2* |
| lavage | | | | | | |
| fluid (×10⁹) | | | | | | |
| Number of | 5.3 ± 1.5 | 20.7 ± 2.5 | 3.3 ± 2.3* | 7.3 ± 0.6* | 3.0 ± 1.7* | 4.0 ± 1.0* |
| inflammatory | | | | | | |
| eosinophils | | | | | | |
| in pneumonic | | | | | | |
| tissue | | | | | | |

*P < 0.01, comparing with the non-treatment group

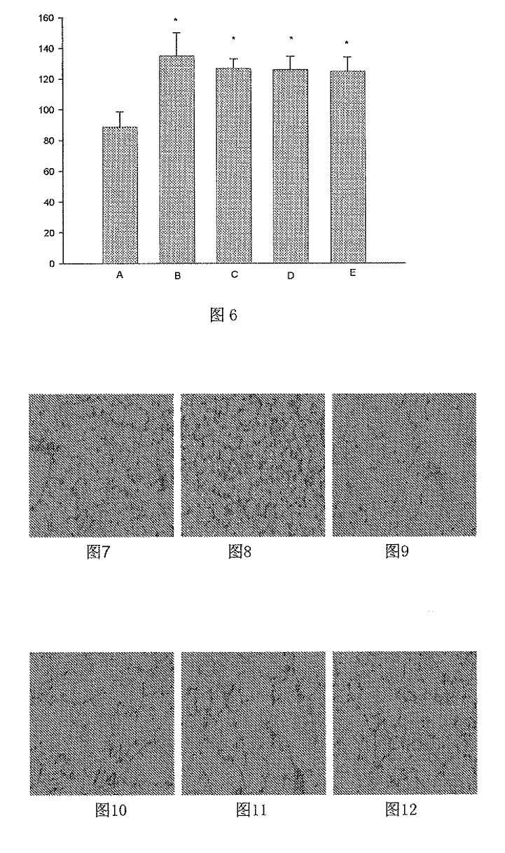

FIGS. 7 to 12 show the infiltration of pneumonic tissue with eosinophils in every group (magnify 200×).

2.7 Glycoprotein Extract from Limax for Treatment of Bronchitis Induced by Ovalbumin

Preparation of model guinea pig with bronchitis asthma and divide to six groups are following the method shown in section 2.6.

### Bronchoalveolar Lavage Experiment of Bronchia

The isobarical lavage machine is infused with Locke's solution, 37° C., the level of bottom is 60-100 cm higher than the T pipe. Guinea pigs are narcosis under intraperitoneal anesthesia by 1.5 ml 1% pentobarbital. Take 2 ml blood from cervical artery for detection of inflammatory factors. Kill the animal and open its chest, separate and cut down the trachea, and then bring out the trachea and the lung together, place in the Locke's solution, 37° C. Extrude the air out of the lung, and ligate the trachea with sleeve and connect with the T pipe. Open the T pipe to let Locke's solution flow into the lung, adjust the velocity of flow to about 25 ml/min. Take 5 ml lavage fluid for detection of inflammatory factors. When the velocity of flow is stable, animals are given 0.5% ovalbumin to stimulate the twitching of bronchia. After 30 minutes, observe and record the flux per minute, 6-10 minutes.

Statistic treatment: data is expressed by x±s, and analyzed by one-way ANOVA, and compare with each group by Newman-Keuls method, P<0.05 meants distinct difference.

### Result of Experiment:

### Prevention of Reduction of Amount of Lung Overfall of Bronchitis Induced by Ovalbumin

There are animals found death during the experiment in every group. The final animals in the experiment are 15 animals. Before stimulating by ovalbumin, the amount of lung overfall of model group is obviously lower than other groups. After stimulating by ovalbumin, the lung overfall of normal control group has no change, but the amount of lung overfall of model group is obviously reduced. Comparing with the model group, the amount of lung overfall of aminophylline group and middle dosage group and high dosage group is obviously increased. The effect of low dosage group is unconspicuous. Data is shown in table 7.

TABLE 7
Effect of lung overfall of bronchitis before and after induction

| | | Before | | | | | | |
|---|---|---|---|---|---|---|---|---|
| | | stimulating by | After stimulating by ovalbumin (min) | | | | | |
| | Examples | ovalbumin (min) | 1 | 2 | 3 | 4 | 5 | 6 |
| Normal | 15 | 22.8 ± 3.1 | 20.3 ± 4.0 | 19.8 ± 4.9 | 19.8 ± 3.2 | 19.7 ± 4.1 | 20.1 ± 5.2 | 22.1 ± 2.2 |
| control group | | | | | | | | |
| Model group | 15 | 18.1 ± 3.2 a | 13.2 ± 2.2 b, e | 12.3 ± 3.0 b, e | 10.0 ± 2.2 b, e | 8.8 ± 1.8 b, e | 8.5 ± 1.4 b, e | 8.5 ± 2.0 b, e |
| Low dosage | 15 | 21.8 ± 4.3 c | 16.8 ± 2.5 c | 14.0 ± 3.1 | 11.6 ± 4.3 | 10.1 ± 3.2 | 9.1 ± 2.3 | 8.9 ± 2.1 |
| group | | | | | | | | |
| Middle dosage | 15 | 23.0 ± 2.1 c | 22.1 ± 1.5 d | 23.0 ± 1.0 d | 22.2 ± 1.4 d | 24.1 ± 1.2 d | 23.6 ± 1.6 d | 22.3 ± 1.6 d |
| group | | | | | | | | |
| High dosage | 15 | 23.5 ± 1.9 c | 22.5 ± 1.2 d | 20.1 ± 2.1 d | 21.5 ± 2.0 d | 21.5 ± 2.7 d | 21.5 ± 2.3 d | 22.0 ± 2.1 d |
| group | | | | | | | | |
| Aminophylline | 15 | 22.2 ± 4.1 c | 21.2 ± 2.0 d | 20.1 ± 1.3 d | 19.1 ± 2.3 d | 20.0 ± 2.5 d | 21.3 ± 2.1 d | 22.2 ± 2.0 d |
| group | | | | | | | | |

a P < 0.05,
b P < 0.01, comparing with normal control group;
c P < 0.05,
d P < 0.01, comparing with model group;
e P < 0.01, comparing with before stimulating.

Influence of Lung Overfall of Isolated Bronchitis by Glycoprotein Extract from Limax in Contractive State

As shown in table 7, the amount of lung overfall of isolated bronchitis by glycoprotein extract from limax in contractive state is obviously reduced. After 6-minute stimulating, the lung overfall of isolated bronchitis becomes stable. Dose glycoprotein extract from limax or aininophylline to each group, the result is shown in table 8. After dosing aminophylline, the amount of lung overfall increases obviously; after dosing glycoprotein extract from limax, the amount of lung overfall has no change.

TABLE 8
compare between glycoprotein extract from limax and aminophylline
for lung overfall of isolated bronchitis in contractive state

| | Before dosing | After dosing |
|---|---|---|
| Middle dosage group | 8.26 ± 1.98 | 10.12 ± 2.91 |
| Aminophylline group | 9.45 ± 1.86 | 17.13 ± 3.08* |

*P < 0.05, comparing with before dosing

Influence of IL-2, IL-4 by Glycoprotein Extract from Limax in Broneboalve-Olar Lavage Fluid and Blood Serum

As shown in table 9, in the asthma model group, the amount of IL-2, IL-4 in blood serum and bronchoalve-olar lavage fluid (BALF) increases obviously; in glycoprotein extract group and aininophylline group, the amount of IL-2, IL-4 decreases obviously.

TABLE 9
changes of the amount of IL-2, IL-4 in blood
serum and BALF of guinea pig (pg/ml, x ± s)

| | IL-2 in serum | IL-2 in BALF | IL-4 in serum | IL-4 in BALF |
|---|---|---|---|---|
| Normal control group | 0.79 ± 0.53 | 0.11 ± 0.06 | 1.68 ± 1.35 | 25.71 ± 6.70 |
| Model group | 6.32 ± 3.20 a | 0.30 ± 0.15 a | 7.87 ± 3.48 a | 53.78 ± 23.67 a |
| Middle dosage group | 1.26 ± 0.98 b | 0.12 ± 0.09 b | 1.89 ± 1.46 b | 29.88 ± 11.23 b |
| Aminophylline group | 1.45 ± 0.86 b | 0.13 ± 0.08 b | 1.78 ± 1.56 b | 26.81 ± 7.76 b |

a P < 0.05, comparing with normal control group;
b P < 0.05, comparing with model group.

2.8 Glycoprotein Extract from Limax for Treatment of Acute Bronchitis
2.8.1 Glycoprotein Extract from limax for Treatment of Cough of Acute Bronchitis

Preparation of model mouse with acute bronchitis: choose white mice, 20-25 g each, male or female. Pre-select method: place a utensil with 0.5 g anhydrous sodium sulfite in an 1 L beaker, add 50% vitriol 5 ml into the utensil, and place mice of each group into the beaker after 15 seconds, and take out the mice after 30 seconds. The mouse which starts cough in 3 minutes is an eligible mouse. Record the delitescence of cough of each animal, and record the times of cough of each animal. After feeding three days, divide the eligible mice into five groups: animals in control group are dosed physiological saline 15 ml/kg; animals in positive group are dosed dextromethorphan by oral dosing 15 mg/kg; animals in high dosage group are dosed 300 mg/kg glycoprotein extract from limax by gavage, once every day, 5 days; animals in middle dosage group are dosed 100 mg/kg glycoprotein extract from limax by gavage, once every day, 5 days; animals in low dosage group are dosed 32 mg/kg glycoprotein extract from limax by gavage, once every day, 5 days. During the dosing, observe the whole condition of animals in each group, and give the above SO₂ stimulation to them for 30 seconds in one hour after the last dosing. Record the delitescence of cough of each mouse, and record the times of cough of each animal in 3 minutes. T-test among groups is made, and the comparison data among medicine groups and control groups is shown in table 10.

TABLE 10

| | Number of | | | |
|---|---|---|---|---|
| Group | Animals | Dosage | Delitescence | Times of Cough |
| Model control group | 15 | 15 ml/kg | 15.70 ± 4.74 | 69.90 ± 13.42 |
| Dextromethorphan group | 15 | 15 mg/kg | 30.40 ± 9.91** | 35.20 ± 5.65** |
| High dosage group | 15 | 300 mg/kg | 27.60 ± 11.51** | 45.30 ± 10.13** |
| Middle dosage group | 15 | 100 mg/kg | 25.00 ± 5.12** | 53.40 ± 8.69** |
| Low dosage group | 15 | 33 mg/kg | 23.00 ± 8.88 | 57.60 ± 7.90* |

*p < 0.05,
**p < 0.01, comparing with the physiological saline control group

As shown in table 10, the glycoprotein extract from limax can obviously prolong the delitescence of cough, and reduce the times of cough. Distinct difference is shown in the high dosage group and middle dosage group, comparing with the physiological saline control group. Distinct difference is shown between low dosage group and high dosage group.
2.8.2 Glycoprotein Extract from Limax for Treatment of Mucus Related Conditions of Acute Bronchitis

Model-building and divide are carrying out according to the above section 2.8.1. Animals are injected 0.25% phenol red by intraperitoneal dosing (0.3 ml/10 g) in 30 minutes after the last dosing. After 30 minutes, kill the animal and separate its trachea, flush three times by 5% sodium bicarbonate solution (1 ml/times), and then combine the flush liquid. The samples are analyzed by colorimetry, and then put to statistic treatment. The result is shown in table 11.

TABLE 11
Glycoprotein extract from limax for treatment
of mucus related conditions of acute bronchitis

| | Dosage | Number of | Result of colorimetry |
|---|---|---|---|
| Group | (mg/kg) | Animals (n) | (μg/10 ml, X ± S) |
| Control group | | 15 | 8.78 ± 1.65 |
| NH₄Cl group | 500 | 15 | 14.15 ± 1.65* |
| High dosage group | 300 | 15 | 14.26 ± 1.21* |
| Middle dosage group | 100 | 15 | 12.87 ± 1.91 |
| Low dosage group | 33 | 15 | 9.03 ± 1.66 |

*p < 0.01, comparing with the physiological saline control group

As shown in table 11, ammonium chloride has obvious anti-cough effect. It shows distinct difference between the ammonium chloride group and control group. The glycoprotein extract from limax dosing 300 mg/kg has the same effect with ammonium chloride. It shows distinct difference between the glycoprotein extract from limax group and control group.

2.9 Glycoprotein Extract from Limax for Ciliary Movement in Trachea Mucous

Divide the rabbits into five groups: control group; positive medicine group; glycoprotein extract from limax groups in high dosage 300 mg/L, middle dosage 100 mg/L, and low dosage 33 mg/L. Kill the rabbits, separate and cut down the trachea into five segments for the following experiment. The trachea mucous is exposed and washed by Ringer's solution (37° C.), then observe the sample by anatomical lens.

Observe the normal value three times, calculate their mean, and then dose the animals by following reagent: physiological saline, 0.01% chloridize acetylcholine (Ach), glycoprotein extract from limax (high dosage group, middle dosage group, and low dosage group).

### Observation

### (1) Ciliary Movement

| | |
|---|---|
| All ciliary active in field of vision: | +++ |
| ⅓~⅔ ciliary active in field of vision: | ++ |
| Less than ⅓ ciliary active in field of vision: | + |
| All ciliary inactive in field of vision: | −− |

### (2) Moving Velocity

Add an ink into trachea mucous, the ink will move to one way (towards larynx), measure the velocity by micrometer and stopwatch, then calculate the moving velocity of mucus moving (second/2 mm). The result is shown in table 12.

TABLE 12
Glycoprotein extract from limax for ciliary
movement in trachea mucous of rabbits

| | Dosage | Sample | Ciliary | |
|---|---|---|---|---|
| Group | (g/L) | (n) | movement | Moving velocity |
| Control group | | 10 | −− | 19.79 ± 8.81 |
| Ach group | 0.1 | 10 | +++ | 5.36 ± 0.64* |
| High dosage | 500 | 10 | +++ | 6.04 ± 0.94* |
| Middle dosage | 100 | 10 | ++ | 9.97 ± 6.63 |
| Low dosage | 50 | 10 | + | 15.22 ± 9.78 |

*P < 0.01, comparing with control group.

2.10 Study on Antibacterial Effect of Glycoprotein Extract from Limax

2.10.1 select some bacterial related to acute pharyngitis, such as Staphyloccocus aureus Rosenbach, and α-hemolytic streptococcus, inject above bacterial into animals, such as mouse, by intraperitoneal dosing or subcutaneous injection, which will make certain animal death. Dosed before infecting the bacterial, and dosed after infecting the bacterial, the animals will be observed for eating, drinking, and movement.

Divide the animals into six groups as following: control group, model group, positive medicine group (ofloxacin 0.5 g/kg), glycoprotein extract from limax group (high dosage 5 g/kg, middle dosage 1 g/kg, low dosage 0.5 g/kg). Observe and record the eating, drinking, and movement in 4 days as the normal value. Besides of control group, animals in other groups are injected 0.5 ml Staphyloccocus aureus Rosenbach by intraperitoneal dosing or subcutaneous injection In the next day, animals are feed 5 days by gavage, twice every day. Observe the condition of eating, drinking, and movement, and mean growth rates, and then compare with control group. The result by statistic treatment is shown in table 13 and table 14.

TABLE 13
Effect of glycoprotein extract from limax for treatment of infection by
Staphyloccocus aureus Rosenbach

| | | | Normal value | | After infection, eating | | | | |
|---|---|---|---|---|---|---|---|---|---|
| | Examples | Dosage | | Drinking | (g)/dringking (ml), day | | | | |
| | (n) | (mg/kg) | Eating/(g)/ | (ml) | Day 1 | Day 2 | Day 3 | Day 4 | Day 5 |
| Control group | 15 | | 3.6 | 4.6 | 3.5/4.4 | 3.6/4.5 | 4.3/4.4 | 4.0/4.4 | 4.1/4.4 |
| Model group | 15 | | 3.5 | 4.5 | 2.5/2/3* | 2.5/2.2* | 2.0/1.9* | 2.2/2.0* | 2.9/3.1* |
| Ofloxacin | 15 | 500 | 3.5 | 4.4 | 1.9/2.7** | 2.5/3.2** | 3.2/3.0** | 3.6/3.5** | 3.9/4.3** |
| group | | | | | | | | | |
| High dosage | 15 | 500 | 3.6 | 4.6 | 2.1/2.2** | 2.0/3.2** | 2.6/3.2** | 3.3/2.8** | 4.0/4.0** |
| group | | | | | | | | | |
| Middle dosage | 15 | 100 | 3.4 | 4.4 | 2.3/2.5 | 1.8/3.0 | 2.3/2.2 | 2.7/2.5 | 3.7/3.4 |
| group | | | | | | | | | |
| Low dosage | 15 | 50 | 3.5 | 4.4 | 2.0/2.2 | 1.9/3.1 | 2.0/1.8 | 2.4/2.2 | 3.5/3.5 |
| group | | | | | | | | | |

*P < 0.01, comparing with control group;
**P < 0.01, comparing with model group.

TABLE 14
Influence of movement of infected animals by glycoprotein extract from limax

| | Number of | After infection | |
|---|---|---|---|
| Group | animals | Survive | movement |
| Control group | 15 | 15 | Normal |
| Model group | 15 | 13 | Activity reduced obviously, hair erection, |
| | | | cyst formation, node formation |
| Ofloxacin group | 15 | 15 | Activity reduced slightly, normal in dosing |
| | | | 3 days, no cyst and node formation |
| High dosage group | 15 | 15 | Activity reduced slightly, normal in dosing |
| | | | 4 days, only two have little node formation |
| Middle dosage group | 15 | 15 | Activity reduced obviously, 7 animals have |
| | | | cyst and node formation |
| Low dosage group | 15 | 15 | Activity reduced obviously, hair erection, |
| | | | cyst formation, node formation |

As shown in table 13 and table 14, the glycoprotein extract from limax may have certain antibacterial effect

2.10.2 Effect of Glycoprotein Extract from Limax for Experimental Treatment of Infection by α-hemolytic streptococcus In Vivo

Divide the animals into six groups as following: control group, model group, positive medicine group (ofloxacin 0.5 g/kg), glycoprotein extract from limax group (high dosage 300 mg/kg, middle dosage 100 mg/kg, low dosage 33 mg/kg). α-hemolytic streplococcus is injected into animals by intraperitoneal dosing. The other methods are the same as the section 2.10.1. The result is shown in table 15.

TABLE 15
Effect of glycoprotein extract from limax for treatment of infection by α-hemolytic
streptococcus

| | | | Normal value | | After infection, eating (g)/ | | | | |
|---|---|---|---|---|---|---|---|---|---|
| | Examples | Dosage | | Drinking | dringking (ml), day | | | | |
| | (n) | (mg/kg) | Eating/(g)/ | (ml) | Day 1 | Day 2 | Day 3 | Day 4 | Day 5 |
| Control group | 15 | | 3.5 | 4.6 | 3.6/4.5 | 3.7/4.4 | 4.0/4.3 | 4.2/4.5 | 4.3/4.6 |
| Model group | 15 | | 3.6 | 4.5 | 2.3/2.1* | 1.9/1.8* | 2.0/1.8* | 2.5/2.5* | 2.8/2.5* |
| Ofloxacin | 15 | 500 | 3.5 | 4.5 | 2.1/2.3 | 2.3/3.6** | 2.9/3.8** | 3.2/3.8** | 4.5/4.2** |
| group | | | | | | | | | |
| High dosage | 15 | 300 | 3.7 | 4.4 | 2.0/2.0 | 2.1/2.8 | 2.6/3.0** | 3.0/4.0** | 4.5/4.0** |
| group | | | | | | | | | |
| Middle dosage | 15 | 100 | 3.5 | 4.5 | 2.2/2.2 | 1.9/2.0 | 2.4/3.0 | 3.0/3.0 | 3.6/3.3** |
| group | | | | | | | | | |
| Low dosage | 15 | 33 | 3.5 | 4.5 | 2.0/2.3 | 1.8/2.1 | 2.0/2.0 | 2.8/2.7 | 3.0/3.2 |
| group | | | | | | | | | |

*P < 0.01, comparing with control group;
**P < 0.01, comparing with model group.

As shown in table 15, the glycoprotein extract from limax may have certain antibacterial effect.

1. A glycoprotein extract which can be used for treating chronic obstructive pulmonary diseases, said glycoprotein mixture is prepared by the following method:
(1) crushing limax material to dry limax powders, storing for next step;
(2) extracting said dry limax powders by ethanol, then filtrating, removing the supernatant, obtaining a residue I;
(3) eliminating ethanol from said residue I, adding water to extract, then filtrating, obtaining a supernatant I;
(4) precipitating said supernatant I by ethanol, concentrating by the centrifugal, removing the supernatant, and eliminating ethanol from the deposit, freeze drying, then the targeted glycoprotein extract is obtained.

2. The glycoprotein extract which can be used for treating chronic obstructive pulmonary diseases according to claim 1, wherein ethanol in said step (2) is 60% ethanol, said extracting process is following by: extracting said dry limax powders by 60% ethanol overnight, then filtrating, removing the supernatant, obtaining a residue I, repeating the above extracting 1-3 times by 60% ethanol in need.

3. The glycoprotein extract which can be used for treating chronic obstructive pulmonary diseases according to claim 1, wherein ethanol in said step (4) is 60% ethanol.

4. The glycoprotein extract which can be used for treating chronic obstructive pulmonary diseases according to claim 1, wherein adding water to extract in said step (3), then filtrating, obtaining a supernatant I and a residue II, and then extracting the residue II by repeating the step (3), combining the supernatant as supernatant I.

5. The glycoprotein extract which can be used for treating chronic obstructive pulmonary diseases according to claim 1, wherein said limax material in step (1) is selected from fresh limax or frozen limax in whole body.

6. A use of the glycoprotein extract according to claim 1 in preparation of pharmaceutical compositions for the treatment of chronic obstructive pulmonary diseases.

7. A use of the glycoprotein extract according to claim 1 in preparation of antibacterial and antiphlogistic pharmaceutical compositions.

8. The use according to claim 7, wherein said antibacterial pharmaceutical compositions are used to preparing antibacterial agents against acute pharyngitis and/or chronic pharyngitis.

9. A pharmaceutical composition comprising of effective amount of the glycoprotein extract according to claim 1, and pharmaceutically acceptable excipients, thinners, and carriers.

10. The pharmaceutical composition according to claim 9 in the form of: capsule, granulate, tablet, pill, guttate pill, syrup, injection, frozen powder for injection, and spray.
